(19) United States
(12) Patent Application Publication
(10) Pub. No.: US 2006/0142326 A1
(43) Pub. Date: Jun. 29, 2006

(54) Use of pyrazolopyridines for the treatment of cognitive deficits

(76) Inventors: Fabien Schweighoffer, Vincennes (FR); Laurent Desire, Paris (FR); Annelies Resink, Paris (FR); Magali Rouquette, Paris (FR)

Correspondence Address: NIXON & VANDERHYE, PC, 901 NORTH GLEBE ROAD, 11TH FLOOR, ARLINGTON, VA, 22203, US

(21) Appl. No.: 10/560,774

(22) PCT Filed: Jun. 25, 2004
(86) PCT No.: PCT/FR04/01630

§ 371 Date: Dec. 14, 2005

(30) Foreign Application Priority Data
Jun. 27, 2003 (FR) 03/07824

(51) Int. Cl. A61K 31/4745

(52) U.S. Cl. 514/303

(57) ABSTRACT

The invention relates to methods which are used to improve, increase or facilitate the cognition of individuals with neurodegenerative pathologies. More specifically, the invention relates to the use of compounds from the family of pyrazolopyridines in order to improve the cognitive faculties of individuals with neurodegenerative diseases. The invention can be used to improve the condition of individuals with different neurodegenerative diseases and, in particular, Alzheimer's disease and vascular dementia.

### FIELD OF THE INVENTION

This invention relates to the field of biology, genetics and medicine. It relates in particular to new compositions and methods for the treatment of neurodegenerative diseases, and in particular in order to improve, increase or facilitate the cognition of individuals with neurodegenerative diseases. More specifically, the invention relates to the use of compounds from the family of pyrazolopyridines in order to improve the cognitive faculties of individuals with neurodegenerative diseases. The invention can be used to improve the condition of individuals with different neurodegenerative diseases, and in particular, Alzheimer's disease or vascular dementia.

### BACKGROUND TO THE INVENTION

Numerous neurodegenerative diseases have been described as having a component or a stage linked to the phenomenon of apoptosis or programmed cell death. One can cite the neurodegenerative diseases of the central nervous system (for example Amyotrophic Lateral Sclerosis—ALS—, Parkinson's disease, Alzheimer's disease or vascular dementia), as well as the peripheral, in particular ocular, degenerative diseases. These diseases mainly have symptomatic treatments, in particular treatments of the associated inflammatory phenomena, but few treatments for the true causes of these disorders, in particular due to the complexity of the metabolic mechanisms and channels involved, and to the diversity of causative factors.

International patent application No. WO03/016563, filed by the applicant, describes new neurotoxicity molecular targets and new therapeutical approaches for the treatment of neurodegenerative diseases. These approaches are based upon a modulation of the activity or the expression of a type 4 phosphodiesterase.

International application No. PCT/FR04/00366, filed by the applicant, proposes new approaches for treating ocular degenerative diseases based upon a modulation of the activity or the expression of a type 4 phosphodiesterase.

Applications WO01/78709, WO01/81348, WO01/81345 and WO03/045949 relate to the use of pyrazolopyridines in the treatment of certain events associated with neurological diseases, such as the formation of peptidic aggregates (WO01/78709), phosphorylation of TAU protein (WO01/81348) or blockage of the GSK-3 enzyme (WO01/81345 and WO03/045949).

### SUMMARY OF THE INVENTION

This application now relates to new therapeutic strategies for neurodegenerative diseases in which the cognitive functions are altered, as observed in Alzheimer's disease and vascular dementia. These strategies are based upon a modulation of one or more metabolic channels identified by the inventors, which are correlated to the appearance, development and progression of excitotoxicity and apoptosis in the nerve cells, and are particularly relevant in neurodegenerative diseases and cognitive function.

More specifically, this application derives from the display of the advantageous and remarkable properties of compounds of the pyrazolopyridine family, including etazolate, for the treatment of cognitive deficits, in particular those induced by Alzheimer's disease and vascular dementia. This application therefore proposes new therapeutic strategies intended for treating or reducing cognitive problems in patients with neurodegenerative disease.

In general therefore, this invention relates to the use of a compound of the pyrazolopyridine family for the treatment of neurodegenerative diseases, in particular of cognitive deficits associated with neurodegenerative diseases.

Another object of the invention is to use a compound of the pyrazolopyridine family for treating or improving the cognitive deficit in individuals with neurodegenerative disease, in particular Alzheimer's disease or vascular dementia.

A more general aspect of the invention also relates to the use of a modulator of GABA(A) and of free radicals for the preparation of a pharmaceutical composition intended for treating neurodegenerative diseases, in particular Alzheimer's disease and vascular dementia, or cognitive problems or disorders in patients with such diseases.

A particular object of the invention is to use a compound of the pyrazolopyridine family for the preparation of a pharmaceutical composition intended for treating cognitive deficits in patients with neurodegenerative disease.

Another object of the invention is a method for increasing cognition or cognitive perception in patients with neurodegenerative disease, comprising administering to a patient a compound as defined above. Advantageously, the method of the invention furthermore makes it possible to inhibit or reduce neuronal excitotoxicity in neurodegenerative diseases.

Without wishing to be linked by an action mechanism, it would appear that the unexpected and advantageous beneficial action of the compounds according to the invention upon cognitive disorders can be explained by a double impact upon the GABA(A) receptor and the mitochondrion. Indeed, this invention describes the identification, in the brain of pathological patients, of three original molecular events characterised by an alteration of the expression of the mRNA of PDE4, of AKAP1 and GABA(A)RAPL1. These events are correlated in time with the phenomena of excitotoxicity and/or of neuronal death, and demonstrate the existence of alterations to the GABA signalling in relation to cognitive problems. Thus in particular, this invention reveals the existence of alterations to the splicing of the mRNA coding for the epsilon sub-unit of the GABA(A) receptor between mRNA extracted from prefrontal cortex of patients with Alzheimer's disease on the one hand and from mRNA extracted from the same brain region of control individuals of the same age, on the other hand. This discovery is particularly interesting because this protein is involved in the presentation and desensitisation of the GABA(A) receptor, and ageing and the processes linked to age are associated with an increase in the time required for desensitising this GABA(A) receptor. These processes, and in particular cognitive deficits, could therefore be compensated by compounds according to the invention, acting upon the GABA channel and mitochondria.

This invention therefore introduces new elements which are essential for electing the GABA(A) receptor as a therapeutical target for the treatment of Alzheimer's disease and, more generally, of cognitive disorders, and thus makes it possible to understand the biological and therapeutical effects observed when using compounds of the pyrazolopyridine family in the treatment of neurodegenerative diseases, including Alzheimer's disease and vascular dementia, and more specifically for treating cognitive disorders. The results shown in the examples illustrate in particular the effectiveness of these compounds in improving memorisation capacities in animals in an aversive situation.

### DETAILED DESCRIPTION OF THE INVENTION

Excitotoxicity and apoptosis are the two main causes of neuronal death. The multiple apoptosis channels emanate from the mitochondrion, and one of the crucial points for the appearance of apoptosis is, for example, the opening of the mitochondrial transition pore (MTPP). Over-production of free radicals (ROS), due to the dysfunction of the mitochondrion, unbalances regulation of apoptosis and thus induces an increase in vulnerability of the neurons to excitotoxicity.

These two phenomena, the over-production of free radicals and excitotoxicity, play a part in the pathological mechanism involving neuronal death due to age and neurodegenerative diseases such as Alzheimer's disease, vascular dementia, Parkinson's disease and ALS. Indeed, it has been demonstrated that free radicals are at least partly responsible for the deficiencies of old brains. Oxidative stress has been implicated in the progression of Alzheimer's disease, vascular dementia, Parkinson's disease and ALS. Oxidative stress is the result of a homeostasis disorder between the pro-oxidants and the anti-oxidants, and this leads to the generation of toxic free radicals.

The inventors have established a repertoire of RNA splicing alterations in the brain of model SLA animals which are 60 days old, and this was achieved by qualitative differential screening according to the DATAS technique (described in application No. W099/46403). This repertoire was constructed from RNA extracted from brain samples and from the spinal cord, without previously isolating the neurons, such as to take into account the maximum number of alternative splicing events linked to the development of the disease. The repertoire produced in this way contains more than 200 distinct sequences, involving key players in the excitotoxicity phenomenon, such as the potassium channels and the NMDA receptor. The specificity of the sequences which make up this repertoire is certified by the fact that the same qualitative differential analysis of the genetic expression carried out on 90 day old animals ends with a different repertoire, from which are absent in particular the different excitotoxicity markers. Analysis of the splicing modifications confirms that the molecular events are different according to the stage of the disease.

In a particularly interesting and unexpected way, by carrying out DATAS on the RNA of controlled and transgenic, 60 day old animals, it was possible to isolate a fragment of cDNA derived from the mRNA of phosphodiesterase 4B, from AKAP1 protein (“A Kinase Anchoring Protein”) and from GABA(A)RAPL1 protein (“GABA(A) Receptor Associated Protein Like 1”).

The PDE4B protein, capable of hydrolysing AMPc, is involved in the regulation of the intracellular concentration of AMPc. The AKAP1 protein anchors the regulating sub-unit of the kinase A protein (activated by AMPc) to the mitochondrial membrane and regulates the activity of the mitochondrial transition pore. The results obtained show a more pronounced expression of PDE4B in the pathological nerve tissues, linked to a structural modification of the corresponding RNA, in particular to the deletion of a region in the non-coding 3′ part. This result is totally compatible with the presence of destabilisation sequences of mRNA in the sequence identified by DATAS. The deletion of these destabilisation sequences of the mRNA of PDE4B, by splicing or by using alternative polyadenylation sequences, can lead to stabilisation, and therefore to an increase in the expression, of the coding part of this RNA. This event happens specifically in the brain of pathological individuals and not in the control individuals.

Moreover, the identification of a fragment derived from AKAP1 demonstrates the involvement of this protein in the development of the excitotoxicity and neuronal death processes. AKAP1 interacts with the regulating sub-unit of the kinase A protein and with the peripheral benzodiazepine receptor (PBR), which participates in regulating the opening of the mitochondrial transition pore, an opening which characterises implementation of apoptosis. Consequently, the invention suggests that AKAP1 regulates the intervention of the PBR in the phenomena of cell death such as neuronal death.

The identification of a fragment derived from GABA(A)RAPL1 emphasises deregulation of the signalling dependent upon the GABA(A) receptor. This observation is totally compatible with the importance of the neurotransmitter as an inhibitor of synaptic transmission, in particular by its interaction with the GABA(A) receptor. This inhibition makes it possible to protect the neurons against sustained excitation which could lead to neuronal death by excitotoxicity. Our work therefore indicates an alteration of this level of regulation, involved in the presentation and the desensitisation of the GABA(A) receptor.

More specifically, the discovery described by this invention illustrates the existence of alterations to the GABA signalling in relation to cognitive problems. This invention also reveals the existence of splicing alterations of the mRNA coding for the epsilon sub-unit of the GABA(A) receptor between mRNA extracted from the prefrontal cortex of patients with Alzheimer's disease on the one hand and from mRNA extracted from the same region of the brain of control individuals of the same age, on the other hand. No anomaly of this sub-unit had ever been reported before now in human pathology.

This invention therefore makes it possible to propose new therapeutical strategies for cognitive disorders, based upon a modulation of these metabolic channels which are correlated to the appearance, development and progression of excitotoxicity and apoptosis in the nerve cells, and are particularly relevant in neurodegenerative diseases and cognitive function.

As indicated above, this invention relates generally to the use of a compound from the pyrazolopyridine family for the treatment of neurodegenerative diseases (including vascular dementia), and in particular of cognitive deficits associated with neurodegenerative diseases.

This application documents the advantageous and remarkable properties of compounds from the pyrazolopyridine family, including etazolate, for the treatment of cognitive deficits, in particular those induced by Alzheimer's disease and vascular dementia.

Within the context of the invention, the term <<treatment >> designates preventive, curative and palliative treatment, as well as the care of patients (reduction of suffering, improvement of life span, deceleration of the progression of the disease, improvement of neuron survival, protection of neurons against excitotoxicity or apoptosis, etc.), etc. Furthermore, the treatment can be carried out in combination with other agents or treatments, in particular addressing the delayed events of the disease, such as caspase inhibitors or other active compounds.

The invention is particularly adapted to the treatment of cognitive deficits in individuals i.e. to the reduction of these effects and/or to the improvement of cognitive perception in patients.

In the sense of the invention, a compound (or ligand) of the pyrazolopyridine family advantageously designates any compound with the following formula (I), which can be substituted or not, on any of the positions.

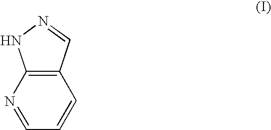

The compounds of the pyrazolopyridine family used in this invention are in particular chosen from the following compounds:

Etazolate with the following formula (II): 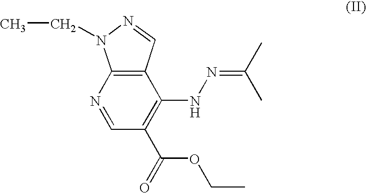
etazolate being a preferred embodiment of the invention,

Ethylic ester 4-butylamino-1-ethyl-6-methyl-1H-pyrazolo[3,4-b]pyridine-5-carboxylic acid (tracazolate),

Ethylic ester of 4-butylamino-1-ethyl-1H-pyrazolo[3,4-b]pyridine-5-carboxylic acid

1-(4-amino-pyrazolo[3,4-b]pyridin-1-yl)-β-D-1-deoxy-ribofuranose

Ethylic ester of 1-ethyl-4-(N′-isopropylidene-hydrazino)-1H-pyrazolo[3,4-b]pyridine-5-carboxylic acid (SQ 20009),

4-amino-6-methyl-1-n-pentyl-1H-pyrazolo[3,4-b]pyridine

Ethylic ester of 4-Amino-1-ethyl-6-methyl-1H-pyrazolo[3,4-b]pyridine-5-carboxylic acid (desbutyl tracacolate),

4-amino-1-pentyl-1H-pyrazolo[3,4-b]pyridine-5-carboxamide,

Ethylic ester of 1-ethyl-6-methyl-4-methylamino-1H-pyrazolo[3,4-b]pyridine-5-carboxylic acid,

Ethylic ester of 4-amino-6-methyl-1-propyl-1H-pyrazolo[3,4-b]pyridine-5-carboxylic acid,

Ethylic ester of 1-ethyl-4-ethylamino-6-methyl-1H-pyrazolo[3,4-b]pyridine-5-carboxylic acid,

Ethylic ester of 4-amino-1-butyl-6-methyl-1H-pyrazolo[3,4-b]pyridine-5-carboxylic acid,

5-(4-amino-pyrazolo[3,4-b]pyridin-1-yl)-2-hydroxymethyl-tetrahydro-furan-3-ol,

allylic ester of 1-allyl-4-amino-6-methyl-1H-pyrazolo[3,4-b]pyridine-5-carboxylic acid,

4-amino-6-methyl-1-pentyl-1H-pyrazolo[3,4-b]pyridine-5-carboxylic acid,

ethylic ester of 4-amino-1-ethyl-3,6-dimethyl-1H-pyrazolo[3,4-b]pyridine-5-carboxylic acid,

ethylic ester 4-dimethylamino-1-ethyl-6-methyl-1H-pyrazolo[3,4-b]pyridine-5-carboxylic acid,

ethylic ester 1-ethyl-6-methyl-4-propylamino-1H-pyrazolo[3,4-b]pyridine-5-carboxylic acid,

ethylic ester 4-amino-1-pentyl-1H-pyrazolo[3,4-b]pyridine-5-carboxylic acid,

ethylic ester of 4-amino-6-methyl-1-pent-4-ynyl-1H-pyrazolo[3,4-b]pyridine-5-carboxylic acid,

4-amino-1-but-3-enyl-1H-pyrazolo[3,4-b]pyridine-5-allylamide,

4-amino-1-pentyl-1H-pyrazolo[3,4-b]pyridine-5-isopropylamide,

4-amino-1-pentyl-N-n-propyl-1H-pyrazolo-[3,4-b]pyridine-5-carboxamide,

allylic ester of 4-amino-1-butyl-6-methyl-1H-pyrazolo[3,4-b]pyridine-5-carboxylic acid,

ethylic ester of 4-amino-6-methyl-1-pent-3-ynyl-1H-pyrazolo[3,4-b]pyridine-5-carboxylic acid,

4-amino-1-pentyl-1H-pyrazolo[3,4-b]pyridine-5-prop-2-ynylamide

allylic ester of 4-amino-1-(3-methyl-butyl)-1H-pyrazolo[3,4-b]pyridine-5-carboxylic acid,

4-amino-1-pentyl-1H-pyrazolo[3,4-b]pyridine-5-N-(2-propenyl)carboxamide,

allylic ester of 4-amino-1-pentyl-1H-pyrazolo[3,4-b]pyridine-5-carboxylic acid,

4-amino-1-pentyl-1H-pyrazolo[3,4-b]pyridine-5-butylamide,

allylic ester of 4-amino-1-but-3-ynyl-6-methyl-1H-pyrazolo[3,4-b]pyridine-5-carboxylic acid,

allylic ester of 4-amino-1-but-3-enyl-6-methyl-1H-pyrazolo[3,4-b]pyridine-5-carboxylic acid,

4-amino-6-methyl-1-pentyl-1H-pyrazolo[3,4-b]pyridine-5-allylamide,

allylic ester of 4-amino-6-methyl-1-pentyl-1H-pyrazolo[3,4-b]pyridine-5-carboxylic acid,

allylic ester of 4-amino-6-methyl-1-(3-methyl-butyl)-1H-pyrazolo[3,4-b]pyridine-5-carboxylic acid,

isobutylic ester of 4-amino-6-methyl-1-pentyl-1H-pyrazolo[3,4-b]pyridine-5-carboxylic acid,

4-amino-6-methyl-1-pentyl-1H-pyrazolo[3,4-b]pyridine-5-butylamide,

allylic ester of 4-amino-6-methyl-1-(3-methyl-but-2-enyl)-1H-pyrazolo[3,4-b]pyridine-5-carboxylic acid,

4-amino-1-pentyl-1H-pyrazolo[3,4-b]pyridine-5-cyclopropylamide,

ethyl 4-amino-1-pentyl-1H-pyrazolo[3,4-b]pyridine-5-hydroxamate,

prop-2-ynylic ester of 4-amino-6-methyl-1-pentyl-1H-pyrazolo[3,4-b]pyridine-5-carboxylic acid,

allylic ester of 4-amino-6-methyl-1-pent-4-ynyl-1H-pyrazolo[3,4-b]pyridine-5-carboxylic acid,

allylic ester of 4-amino-6-methyl-1-pent-4-enyl-1H-pyrazolo[3,4-b]pyridine-5-carboxylic acid,

4-amino-1-pent-3-ynyl-1H-pyrazolo[3,4-b]pyridine-5-propylamide,

4-amino-1-pentyl-1H-pyrazolo[3,4-b]pyridine-5-cyclopropylmethyl-amide,

2-methyl-allylic ester of 4-amino-6-methyl-1-pentyl-1H-pyrazolo[3,4-b]pyridine-5-carboxylic acid,

4-Amino-1-pent-3-ynyl-1H-pyrazolo[3,4-b]pyridine-5-allylamide (ICI 190,622),

4-amino-1-pent-4-ynyl-N-2-propenyl-1H-pyrazolo[3,4-b]pyridine-5-carboxamide,

4-amino-1-pent-3-ynyl-1H-pyrazolo[3,4-b]pyridine-5-prop-2-ynylamide,

4-amino-1-pentyl-1H-pyrazolo[3,4-b]pyridine-5-but-2-ynylamide,

allylic ester of 4-amino-6-methyl-1-pent-3-ynyl-1H-pyrazolo[3,4-b]pyridine-5-carboxylic acid,

allylic ester of 4-amino-1-(2-cyclopropyl-ethyl)-6-methyl-1H-pyrazolo[3,4-b]pyridine-5-carboxylic acid,

allylic ester of 4-amino-1-hex-5-ynyl-6-methyl-1H-pyrazolo[3,4-b]pyridine-5-carboxylic acid,

4-amino-1-pent-3-ynyl-1H-pyrazolo[3,4-b]pyridine-5-cyclopropylmethyl-amide,

but-3-enylic ester of 4-amino-6-methyl-1-pentyl-1H-pyrazolo[3,4-b]pyridine-5-carboxylic acid,

cyclopropylmethylic ester of 4-amino-6-methyl-1-pentyl-1H-pyrazolo[3,4-b]pyridine-5-carboxylic acid,

4-butylamino-1-pentyl-1H-pyrazolo[3,4-b]pyridine-5-allylamide,

2-cyclopropyl-ethylic ester of 4-amino-6-methyl-1-pentyl-1H-pyrazolo[3,4-b]pyridine-5-carboxylic acid,

cyclopropylmethylic ester of 4-amino-6-methyl-1-pent-3-ynyl-1H-pyrazolo[3,4-b]pyridine-5-carboxylic acid,

cyclopropylmethylic ester of 4-amino-6-methyl-1-pent-4-ynyl-1H-pyrazolo[3,4-b]pyridine-5-carboxylic acid,

ethylic ester of 4-amino-1-benzyl-6-methyl-1H-pyrazolo[3,4-b]pyridine-5-carboxylic acid,

4-amino-1-pentyl-1H-pyrazolo[3,4-b]pyridine-5-benzylamide,

4-amino-1-pentyl-1H-pyrazolo[3,4-b]pyridine-5-phenylamide,

benzylic ester of 4-amino-6-methyl-1-pentyl-1H-pyrazolo[3,4-b]pyridine-5-carboxylic acid,

4-Azido-1-β-D-ribofuranosylpyrazolo[3,4-b]pyridine,

1-pent-3-ynyl-N-2-propenyl-4-propionamido-1H-pyrazolo[3,4-b]pyridine-5-carboxamide,

2-(4-amino-pyrazolo[3,4-b]pyridin-1-yl)-5-hydroxymethyl-tetrahydro-furan-3,4-diol,

2-(6-methyl-1H-pyrazolo[3,4-b]pyridin-4-ylamino)-ethanol,

3-(6-methyl-1H-pyrazolo[3,4-b]pyridin-4-ylamino)-propan-1-ol,

propylic ester of 3-(6-methyl-1H-pyrazolo[3,4-b]pyridin-4-ylamino)-acetic acid,

ethylic ester of 2-(6-methyl-1H-pyrazolo[3,4-b]pyridin-4-ylamino)-propionic acid,

ethylic ester of 2-(6-methyl-1H-pyrazolo[3,4-b]pyridin-4-ylamino)-pentanoic acid,

ethylic ester of 2-(6-methyl-1H-pyrazolo[3,4-b]pyridin-4-ylamino)-benzoic acid,

propylic ester of 3-(6-methyl-1H-pyrazolo[3,4-b]pyridin-4-ylamino)-pentanoic acid,

N-benzylidene-N′-(3-methyl-1-phenyl-1H-pyrazolo[3,4-b]pyridin-4-yl)-hydrazine,

N-furan-2-ylmethylene-N′-(3-methyl-1-phenyl-1H-pyrazolo[3,4-b]pyridin-4-yl)-hydrazine,

N-(4-fluoro-benzylidene)-N′-(3-methyl-1-phenyl-1H-pyrazolo[3,4-b]pyridin-4-yl)-hydrazine,

N-(3-furan-2-yl-allylidene)-N′-(3-methyl-1-phenyl-1H-pyrazolo[3,4-b]pyridin-4-yl)-hydrazine,

N-(4-methoxy-benzylidene)-N′-(3-methyl-1-phenyl-1H-pyrazolo[3,4-b]pyridin-4-yl)-hydrazine,

4-[(3-methyl-1-phenyl-1H-pyrazolo[3,4-b]pyridin-4-yl)-hydrazonomethyl]-benzonitrile,

N-benzo[1,3]dioxol-5-ylmethylene-N′-(3-methyl-1-phenyl-1H-pyrazolo[3,4-b]pyridin-4-yl)-hydrazine,

N-(3-methyl-1-phenyl-1H-pyrazolo[3,4-b]pyridin-4-yl)-N′-(4-nitro-benzylidene)-hydrazine,

N-(3-methyl-1-phenyl-1H-pyrazolo[3,4-b]pyridin-4-yl)-N′-(2-nitro-benzylidene)-hydrazine,

N-(3-methyl-1-phenyl-1H-pyrazolo[3,4-b]pyridin-4-yl)-N′-(4-trifluoromethyl-benzylidene)-hydrazine,

N-(3-methyl-1-phenyl-1H-pyrazolo[3,4-b]pyridin-4-yl)-N′-(5-nitro-furan-2-ylmethylene)-hydrazine,

N-(3-methyl-1-phenyl-1H-pyrazolo[3,4-b]pyridin-4-yl)-N′-(2-trifluoromethyl-benzylidene)-hydrazine,

N-(3-methyl-1-phenyl-1H-pyrazolo[3,4-b]pyridin-4-yl)-N′-(6-nitro-benzo[1,3]dioxol-5-ylmethylene)-hydrazine,

4-(3-chloro-4-methoxy-benzylamino)-1-ethyl-1H-pyrazolo[3,4-b]pyridine-5-carboxylic acid,

4-(3-chloro-4-methoxy-benzylamino)-1-ethyl-1H-pyrazolo[3,4-b]pyridine-5-(pyridin-4-ylmethyl)-amide,

4-(3-chloro-4-methoxy-benzylamino)-1-ethyl-1H-pyrazolo[3,4-b]pyridine-5-(tetrahydro-furan-2-ylmethyl)-amide,

4-(3-chloro-4-methoxy-benzylamino)-1-ethyl-1H-pyrazolo[3,4-b]pyridine-5-(5-hydroxy-pentyl)-amide,

4-(3-chloro-4-methoxy-benzylamino)-1-ethyl-1H-pyrazolo[3,4-b]pyridine-5-[3-(2-oxo-pyrrolidin-1-yl)-propyl]-amide,

ethylic ester of 4-tert-butylamino-1-(2-chloro-2-phenyl-ethyl)-1H-pyrazolo[3,4-b]pyridine-5-carboxylic acid,

ethylic ester of 1-(2-chloro-2-phenyl-ethyl)-4-cyclopropylamino-1H-pyrazolo[3,4-b]pyridine-5-carboxylic acid,

ethylic ester of 1-(2-chloro-2-phenyl-ethyl)-4-propylamino-1H-pyrazolo[3,4-b]pyridine-5-carboxylic acid,

ethylic ester of 1-(2-chloro-2-phenyl-ethyl)-4-phenylamino-1H-pyrazolo[3,4-b]pyridine-5-carboxylic acid,

ethylic ester of 4-butylamino-1-(2-chloro-2-phenyl-ethyl)-1H-pyrazolo[3,4-b]pyridine-5-carboxylic acid,

ethylic ester of 1-(2-chloro-2-phenyl-ethyl)-4-(2-ethoxy-ethylamino)-1H-pyrazolo[3,4-b]pyridine-5-carboxylic acid,

ethylic ester of 4-benzylamino-1-(2-chloro-2-phenyl-ethyl)-1H-pyrazolo[3,4-b]pyridine-5-carboxylic acid,

ethylic ester of 1-(2-chloro-2-phenyl-ethyl)-4-phenethylamino-1H-pyrazolo[3,4-b]pyridine-5-carboxylic acid.

The compounds can be in the form of salt, ester, racemate, active isomer, etc. The capacity of the compounds to protect the free radical cells can be checked in vitro. A particularly preferred compound is etazolate, tracazolate or cartazolate, and more preferably etazolate.

This invention thus proposes, for the first time, a therapeutical intervention linking a modulation of the free radicals and a modulation of the GABA(A) receptor as a therapeutical target for the treatment of cognitive deficits associated with neurodegenerative diseases. According to particular embodiments, the invention can be used to treat the cognitive deficits in the premature phase of these diseases. It is applicable in particular in the case of Alzheimer's disease, vascular dementia, Huntington's chorea and Parkinson's disease.

A particular object of the invention is the use of a pyrazolopyridine compound for the preparation of a medication for treating the cognitive deficit in patients with Alzheimer's disease and vascular dementia.

Another object of the invention is the use of a pyrazolopyridine compound, in particular etazolate, for the preparation of a medication for treating cerebral ischaemia.

Another object of the invention relates to the use of a modulator of the GABA(A) and/or of free radicals for the preparation of a pharmaceutical composition intended for the treatment of neurodegenerative diseases, in particular Alzheimer's disease and vascular dementia, or cognitive problems or disorders in patients with such diseases. The modulator compound can be any chemical compound, natural or synthetic in origin, in particular an organic or inorganic molecule, with a plant, bacterial, viral, animal, eukaryotic, synthetic or semi-synthetic origin, capable of modulating the expression or the activity of the free radicals (ROS).

The compounds used within the framework of this invention can be formulated and administered in different ways. The administration can be carried out by any method known to experts in the field, preferably orally or by injection, which is systemic or local. The injection is typically administered via the intra-ocular, intra-peritoneal, intra-cerebral, intravenous, intra-arterial, sub-cutaneous or intra-muscular route. Administration via the oral or systemic route is preferred. The doses administered can be adapted by the expert. Typically, between approximately 0.01 mg and 100 mg/kg are injected, for compounds which are chemical in nature. Specific unitary doses are for example between 0.5 and 40 mg per dose administered. It goes without saying that repeat injections can be administered, possibly in combination with other active agents or any vehicle which is pharmaceutically acceptable (e.g., buffers, saline solutions, isotonic, in the presence of stabilising agents, etc.).

The pharmaceutically acceptable vehicle or excipient can be chosen from buffer, solvent, binding, stabilising, emulsifying solutions, etc. Buffer or thinning solutions are in particular calcium phosphate, calcium sulphate, lactose, cellulose, kaolin, mannitol, sodium chloride, starch, caster sugar and hydroxy-propyl methyl cellulose (HPMC) (for delayed liberation). Binders are for example starch, gelatine and packing solutions such as sucrose, glucose, dextrose, lactose, etc. Natural or synthetic gums can also be used, such as in particular alginate, carboxymethyl cellulose, methyl cellulose, polyvinyl pyrrolidone, etc. Other excipients are for example cellulose and magnesium stearate. Stabilising agents can be incorporated into the formulations, such as for example polysaccharides (acacia, agar, alginic acid, guar gum and tragacanth, chitin or its derivatives and cellulose ethers). Solvents or solutions are for example Ringer's solution, water, distilled water, phosphate buffers, phosphated saline solutions, and other conventional fluids.

The invention can be used in mammals, in particular in human beings. The results shown in the examples illustrate the effectiveness of etazolate for improving the viability of neurons placed under conditions of excitotoxicity, oxidative stress or cerebral ischaemia, and for improving the memorisation capacities of animals in an aversive situation.

The invention also makes it possible to develop tests, kits or detection processes, screening or to diagnose these diseases in vitro, based upon establishing the presence of deregulation or alteration in a gene, a messenger or a PDE4 or AKAP1 or GABA(A)RAPL1 protein, in an individual. The invention also provides tools for implementing such tests, in particular probes, primers, cells, reagents, etc.

The invention also provides tests or processes for screening candidate molecules for the treatment of neurodegenerative diseases, including establishing the capacity of molecules to bind AKAP1, GABA(A)RAPL1, the GAB(A) receptor and/or PDE4, in particular the altered forms of these genes or proteins as described above.

Other aspects and advantages of this invention will become clear from reading the following examples, which must be considered as illustrative and not limiting.

### LEGEND TO THE FIGURES

FIG. 1: Neuroprotective effect of etazolate upon toxicity induced by NMDA/serine on granular cells of the cerebellum.

FIG. 2: Neuroprotective effect of etazolate upon toxicity induced by kainate on granular cells of the cerebellum.

FIG. 3: Neuroprotective effect of etazolate upon toxicity induced by NMDA/serine on cortical neurons.

FIG. 4: Neuroprotective effect of etazolate upon toxicity induced by kainate on cortical neurons.

FIG. 5: Neuroprotective effect of etazolate upon toxicity induced by NMDA/serine on ventral spinal cord cells.

FIG. 6: Neuroprotective effect of etazolate upon toxicity induced by 6-hydroxydopamine on SH-SY5Y cells

FIG. 7: Protective effect of etazolate in the cerebral infarction model in a rat.

### EXAMPLES

### Example 1

### Identification of PDE4, AKAP1 and GABA(A)RAPL1 as Molecular Excitotoxicity Targets

The differential qualitative analysis was carried out using polyadenylated RNA (poly A+) extracted from samples of animal brains corresponding to the different stages, without previously isolating the neurons so as to take into account the maximum number of alternative splicing events linked to the development of the disease.

The poly A+ RNA are prepared according to techniques known to experts in the field. In particular this can be a treatment using chaotropic agents such as guanidium thiocyanate followed by an extraction of the total RNA using solvents (phenol, chloroform for example). These methods are well known to experts in the field (see Maniatis et al., Chomczynsli et al., Anal. Biochem. 162 (1987) 156), and can easily be put into practice by using the commercially available kits. Starting with these total RNA, the poly A+ RNA are prepared according to classic methods known to experts in the field and proposed by commercial kits.

These poly A+ RNA serve as a matrix for reverse transcription reactions with the help of reverse transcriptase. Advantageously reverse transcriptases are used which have no RNase H activity which make it possible to obtain first complementary DNA strands which are larger in size than those obtained with classic reverse transcriptases. These reverse transcriptase preparations without RNase H activity are available commercially.

For each point of the development kinetics of the disease (30 days, 60 days and 90 days) the poly A+ RNA and the single strand cDNA are prepared from transgenic animals (T) and from syngenic control animals (C).

In accordance with the DATAS technique, for each point of the kinetics, one realises hybridisations of mRNA (C) with cDNA (T) and reciprocal hybridisations of mRNA (T) with cDNA (C).

These mRNA/cDNA heteroduplexes are then purified according to the protocols from the DATAS technique.

The RNA sequences which are not paired with a complementary DNA are liberated from these heteroduplexes by the action of RNase H, this enzyme degrading the paired RNA sequences. These unpaired sequences represent the qualitative differences which exist between RNA which are otherwise homologous with one another. These qualitative differences can be located anywhere on the RNA sequence, at 5′, 3′ or within the sequence and notably in the coding sequence. According to their location, these sequences can be not only splicing modifications but also consequences of translocations or deletions. The RNA sequences representing the qualitative differences are then cloned according to the techniques known to experts in the field, and in particular those described in the DATAS technique patent.

These sequences are regrouped within cDNA banks which are differential qualitative banks. One of these banks contains the specific exons and introns of the healthy situation; the other banks contain the splicing events which are characteristic of the pathological conditions.

The differential expression of the clones was verified by hybridisation with probes obtained by reverse-transcription from messenger RNA extracted from the different situations studied. The clones hybridising differentially were kept for subsequent analysis. The sequences identified by DATAS correspond to introns and/or exons expressed differentially by splicing between the pathological situations and the healthy situation. These splicing events can be specific to a given stage of the development of the disease or characteristic of the healthy state.

Comparison of these sequences with the data banks makes it possible to classify the information obtained and to propose a reasoned selection of sequences according to their diagnostic or therapeutic interest.

By carrying out DATAS on the RNA of controlled and transgenic 60 day old animals, it was possible to isolate a fragment of cDNA derived from the mRNA of phosphodiesterase 4B. This fragment corresponds to a fragment of exon specifically present in the control animals and so specifically deleted in the transgenic animals for SOD1G93A at the 60 day stage. This fragment covers nucleotides 377 to 486 referenced from the stop codon of PDE4B from mice (sequence accessible in GenBank, No. AF208023). This sequence includes 2912 bases, the deleted fragment corresponding to bases 2760 to 2869. This region is non-coding and is expressed differentially between the control animals and the transgenic animals, because of the alternative use of a 3′ non-coding exon or because of the use of two alternative polyadenylation sites.

By carrying out DATAS on the RNA of control and transgenic 60 day old animals, it was also possible to isolate a fragment of cDNA derived from the mRNA of AKAP1. This fragment corresponds to a fragment of exon specifically present in the control animals and so specifically deleted in the transgenic animals for SOD1G93A at the 60 day stage. This fragment is homologous with nucleotides 1794 to 2322 of the sequence referenced in GenBank under No. NM_{—}009648. This region is coding and is expressed differentially between the control animals and the transgenic animals, due to alternative splicing.

By carrying out DATAS on the RNA of control and transgenic 60 day old animals, it was also possible to isolate a fragment of cDNA derived from the mRNA of GABA(A)RAPL1. This fragment corresponds to a fragment of exon specifically present in the control animals and so specifically deleted in the transgenic animals for SOD1G93A at the 60 day stage. This fragment is homologous with nucleotides 1055 to 1461 of the sequence referenced in GenBank under No. BC024706. This region is derived from the non-coding 3′ region and is expressed differentially between the control animals and the transgenic animals.

These elements make it possible to elucidate and to define important signalling channels, and show that the signalling dependent upon GABA(A)R seems to be altered in the brain of patients with Alzheimer's disease. Indeed, analysis by qualitative differential screening according to the DATAS technique of mRNA extracted from the prefrontal cortex of patients with Alzheimer's disease on the one hand, and from RNA extracted from the same region of the brain of control individuals of the same age, demonstrated splicing alterations of the mRNA coding for the GABA(A)RAP protein (GABA(A) Receptor Associated Protein). This alteration reveals the retention of 135 bases of an intronic sequence on base 273 of the sequence from the repertoire in GenBank under number NM_{—}007278.1. This retention modifies the open phase and so the functionality of the GABA(A)RAP protein. Because this protein is involved in the presentation and desensitisation of the GABA(A) receptor, the DATAS analysis reveals an alteration at this level of the regulation of the synaptic activities.

GABA signalling represents one of the most powerful mechanisms for the negative regulation of synaptic activity. When this GABA(A) receptor is stimulated by the GABA neuromediator, this receptor, which is an ionic channel, allows the entry of chlorine ions which are involved in the repolarisation of the neurons. The GABA(A) receptor has a pentameric structure formed by the association of 2 alpha sub-units, two beta sub-units and an accessory, mainly delta, epsilon or gamma sub-unit.

The agonists of the GABA(A) receptor are anxiolytic but amnesiant.

The antagonists of the GABA(A) receptor are anxiogenic, proconvulsant and promnesiant.

In addition, it is known that, in the brain of patients with Alzheimer's disease, one of the sub-units of the GABA(A) receptor, the beta3 sub-unit, is under-expressed.

The epsilon sub-unit, present in the hippocampus and which is one of the first cerebral structures to be altered in the development of Alzheimer's disease, gives original pharmacological properties to the GABA(A) receptor. Indeed, the linkage, via the beta sub-units, to the GABA(A) receptors which contain an epsilon sub-unit, of pharmacological agents such as pyrazolopyridines, such as tracazolate and etazolate, accelerates the desensitisation of the GABA(A) receptor following interaction with the GABA neurotransmitter. This effect is particularly interesting because ageing is associated with an increase in the time necessary for desensitisation of the GABA(A) receptor.

An alteration of the epsilon sub-unit, as described in this invention, associated with elongation of the period necessary for desensitisation of the GABA(A) receptors in the processes linked to age such as Alzheimer's disease, can therefore be compensated by the treatment of patients with pharmacological agents, such as pyrazolopyridines, such as tracazolate and etazolate. The latter compound also offers the advantage of being a PDE4 inhibitor, of which the invention shows the involvement in the excitotoxicity phenomena.

The possibility of affecting this signalling channel can lead to particularly effective treatments of neurodegenerative diseases, in particular degenerative diseases associated with an alteration of the cognitive functions such as Alzheimer's disease and vascular dementia.

### Example 2

### Inhibition of Excitotoxicity

In this example, granular neurons of the cerebellum, cortical neurons and cells of the ventral spinal cord of a rat are placed in culture according to the techniques described below.

Primary Culture of Granular Cells of the Cerebellum

Seven day old Wistar rats are decapitated and their cerebella are dissected. After having removed the meninges, the tissue is cut into small pieces and trypsinised for 15 minutes at 37° C. The cells are disassociated by trituration and placed in cultures at a density of 300,000 cells per cm² in Eagle's base medium supplemented with 10% foetal calf serum and 2 mM glutamine. The following day 10 μM ARA-C, an antimitotic, is added so as to prevent proliferation of the glial cells. The cells are treated for 9 days after placing in culture with the etazolate inhibiting compound, before adding toxics, 50 μM kainate or 100 μM N-methyl-D-aspartate in the presence of 10 μM D-serine. 8-bromo-cAMP is added just before the toxics. All of the treatments are carried out at least in duplicate and in at least two different cultures. Following an incubation of six hours, the toxicity is measured by an MTT test. The results, standardised to the untreated average, are statistically analysed by the Wilcoxon test. The significant value is established at p less than or equal to 0.05.

Primary Cultures of the Cortical Cells:

16 day old Wistar rat embryos are taken and the cortexes dissected. Following trypsination at 37° C. for 25 minutes, the cells are dissociated by trituration. The cells are sown in minimum essential medium, supplemented with 10% horse serum and 10% foetal calf serum and 2 mM glutamine, at a density of 300,000 cells per cm². After 4 days in culture half of the medium is changed with minimum essential medium supplemented with 5% horse serum and 2 mM glutamine. On the same day, 10 μM of 5-fluoro-2-deoxyuridine, an antimitotic, is added. After seven and eleven days of culture, half of the medium is changed with conditioned medium. The conditioned medium is made up from MEM containing 5% horse serum and 2 mM glutamine ; this medium is passed over a carpet of cortical astrocytes for a night before being used. On day 14, the cells are treated with the etazolate inhibitor compound, before adding toxics, 50 μM kainate or 20 μM N-methyl-D-aspartate in the presence of 10 μM D-serine. All of the treatments are carried out at least in duplicate and in at least two different cultures. Following an incubation of six hours the toxicity is measured by an MTT test. The results, standardised to the untreated average, are statistically analysed by the Wilcoxon test. The significant value is established at ρ less than or equal to 0.05.

Primary Cultures of Ventral Spinal Cord Cells:

The cells are isolated from 14 day old Wistar rat embryos. Upon their arrival, the pregnant female rats are sacrificed by means of carbon dioxide.

The series of embryos is taken and placed in a box containing PBS.

The spinal cord of each embryo is dissected and the ventral cord is separated from the dorsal cords. The ventral cords are then trypsinised at 37° C. for 20 mins. The effect of the trypsin is halted by the addition of a medium made up from Leibovitz 15 medium, 20% horse serum, N2 (1×) supplement, 20% glucose (3.2 mg/ml), 7.5% bicarbonate (1.8 mg/ml) and L-glutamine (2 mM). The cells are dissociated by trituration. The accumulated tissue is removed and the dissociated cells are then quantified by dying with trypan blue. The cells sown at a density of 250 000 cells/cm² in a medium made up from neurobasal medium, horse serum (2%), B27 (1×) supplement, and glutamine (2 mM). After 3 days of in vitro culture, an anti-mitotic agent, ARA-C (5 μM), is added to the cells so as to inhibit production of glial cells. The cells are placed in culture at 37° C. in a humidified incubator (5% CO2) for 9 days. After 9 days of culture, the cells are treated with the inhibiting compound: etazolate, before adding 25 μM of N-methyl-D-aspartate (NMDA) in the presence of 10 μM D-serine. All of the treatments are carried out at least in duplicate and in at least two different cultures. After 3 hours of incubation with NMDA/D-serine as a toxic, the toxicity is revealed by means of an MTT test.

The results are standardised to the average of the untreated controls and analysed statistically by means of a Wilcoxon test with ρ less than 0.05.

MTT Test:

Toxicity is measured using the MTT test. Following incubation with the compounds, MTT is added at a final concentration of 0.5 mg/ml per well. The plaques are then incubated for 30 minutes at 37° C. in the dark. The medium is aspirated and the crystals are placed back in suspension in 500 μl of DMSO (dimethylsulfoxide). The absorbance at 550 nm is read and the viability percentage is calculated.

Results:

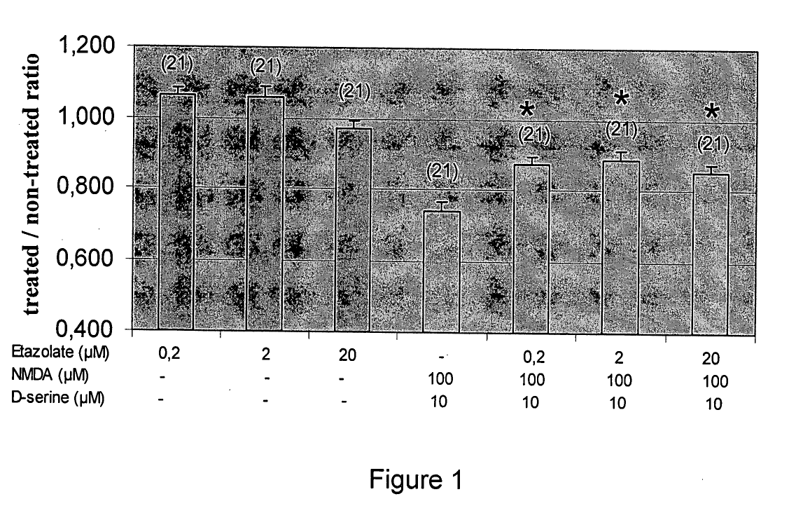

The results obtained are shown in FIGS. 1-5. These results illustrate the protective effect of the compounds of the invention upon neuronal survival. During co-treatment of the neurons by an inhibitor of the invention, a dose-dependent protective effect is observed in the excitotoxicity induction modes (NMDA/Serine and/or kainate).

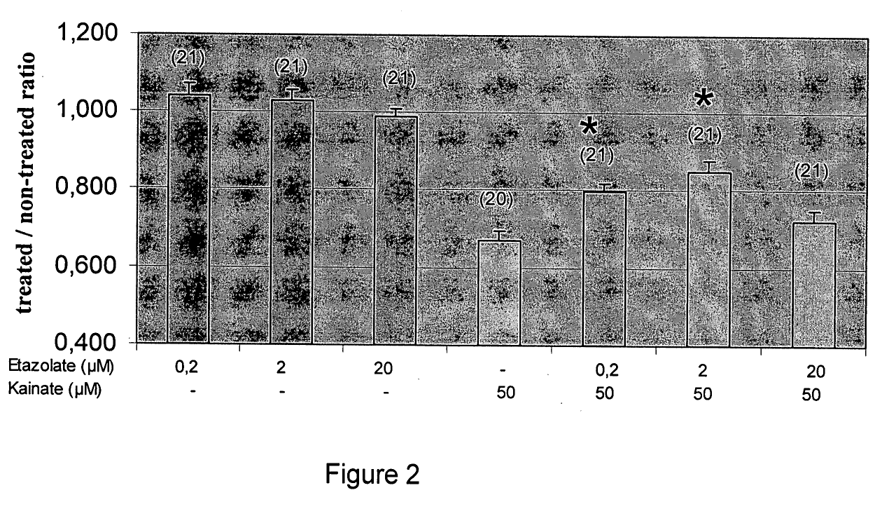

FIGS. 1 and 2 show results obtained with the help of etazolate on the granular cerebellum cells. The results shown demonstrate that etazolate makes it possible to achieve on these cells a 40% protective effect in the case of NMDA/Ser treatment, and 50% in the case of toxicity induced by kainate.

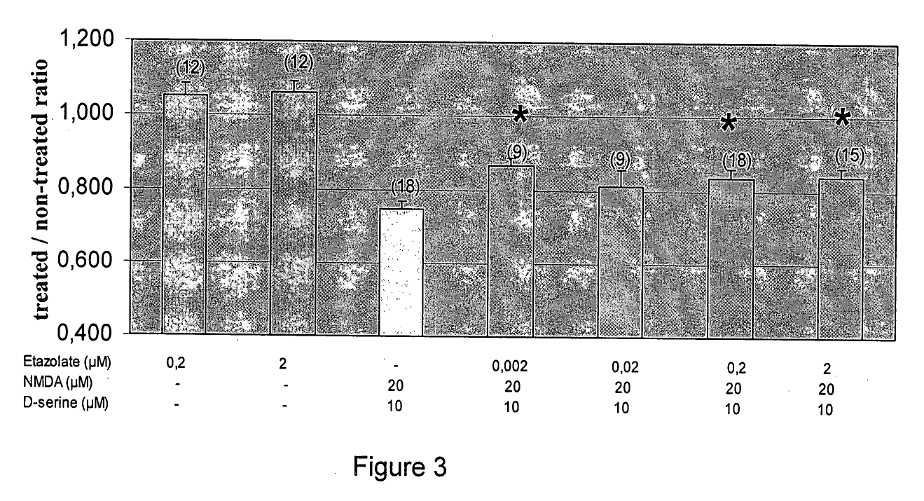

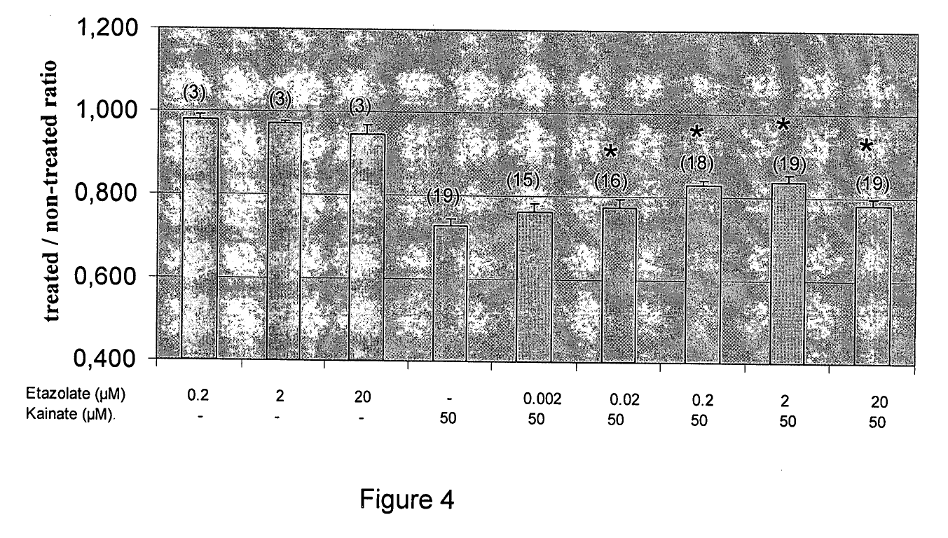

FIGS. 3 and 4 show results obtained with the help of etazolate on cortical neurons. The results shown demonstrate that etazolate makes it possible to achieve on these cells a 47% protective effect in the case of NMDA/Ser treatment, and 40% in the case of toxicity induced by kainate.

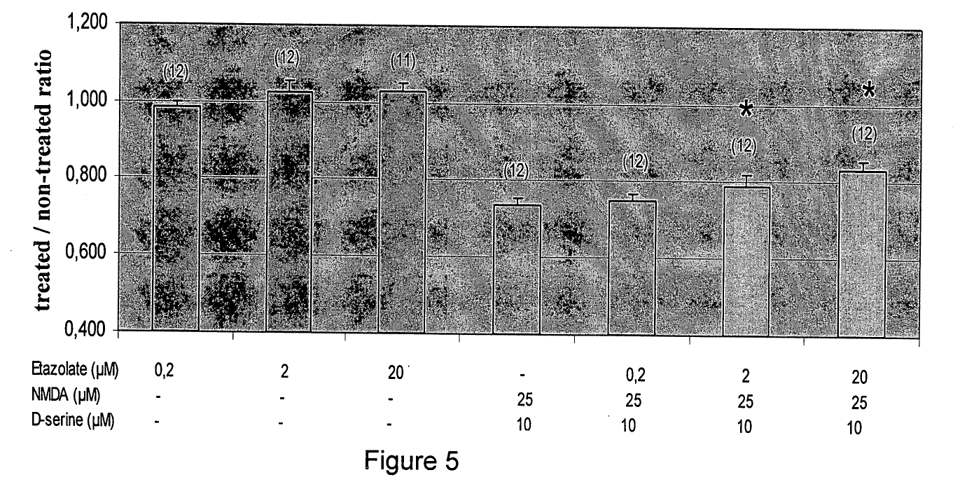

FIG. 5 shows the results obtained with etazolate on ventral spinal cord cells. These results show that etazolate makes it possible to achieve on these cells a 36% protective effect in the case of NMDA/Ser treatment.

This invention documents therefore not only the involvement of PDE4B and the GABA(A) receptors in the excitotoxicity mechanisms, but also the capacity of inhibitors to preserve neuronal viability during stress linked to excitotoxicity.

### Example 3

### Inhibition of Oxidative Stress

In this example, cells from the SH-SY5Y line were placed in culture according to the techniques known to experts in the field. These cells, derived from a human neuroblast, have properties which characterise a neuronal precursor at a premature stage of development.

The toxic used is 6-hydroxydopamine (6-OHDA) which induces oxidative stress. Toxicity is measured by an MTT test.

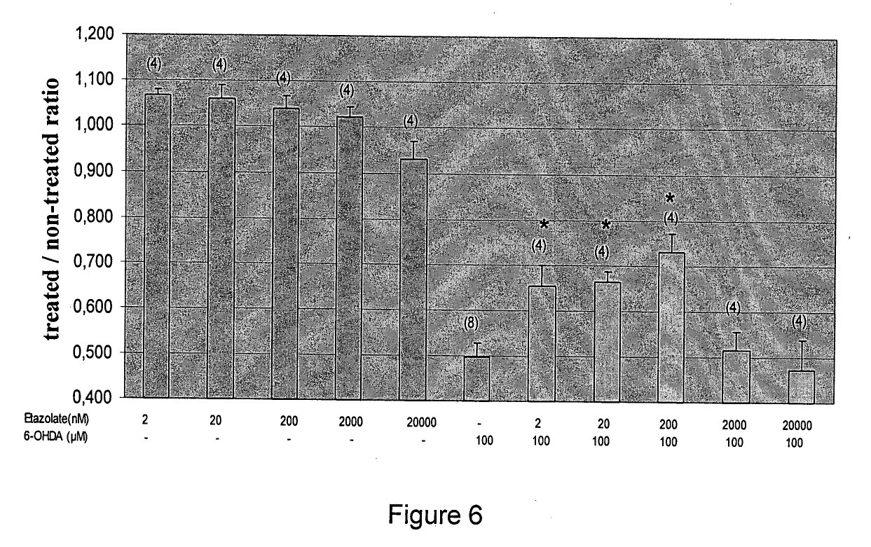

FIG. 6 shows the results obtained with etazolate on SH-SY5Y cells. These results show that etazolate makes it possible to achieve on these cells a 40% protective effect in the case of 6-OHDA treatment.

Etazolate is therefore a potential protector, in vitro, of cell death induced by ROS.

The neuroprotective potential of etazolate is therefore reinforced by the results obtained in examples 2 and 3.

### Example 4

### Study of Ischaemia in Rats

The in vivo protective effect of etazolate was evaluated in a model of cerebral infarction in rats. During this study, a cerebral infarction was brought about by an intracavity occlusion of the internal carotide and the medium arteries of the brain. A group of eight rats was treated with etazolate (10 mg/kg, p.o.) before and several times after occlusion. A group of eight rats was treated with the reference compound, L-NAME (1 mg/kg, i.p.) before and several times after occlusion. A group of eight rats was only treated with the vehicle. The effects were evaluated by clinical observations, neurological function tests and by establishing the size of the infarction at the end of the study.

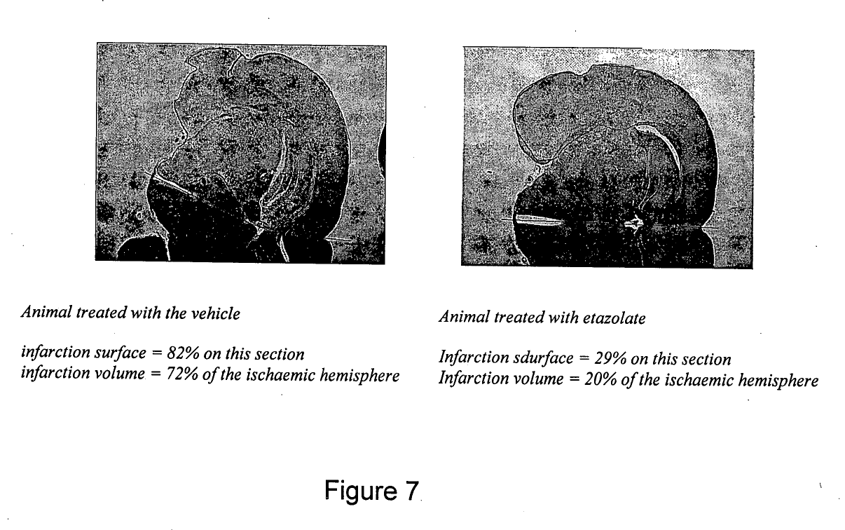

The results obtained show that etazolate induces a reduction averaging 28% of the size of the infarction relative to the control (see example in FIG. 7). On the other hand, an improvement of hypoactivity was observed in the group treated with etazolate relative to the control group (31% for the etazolate group versus 42% for the control group). In addition, the neurological evaluation of animals demonstrates an improvement in the group treated relative to the control group.

### Example 5

### Aquatic Labyrinth Test (Morris Pool)

This test is used to evaluate the capacities to memorise and to manage spatial information in rats in an aversive situation. The task consists for the animal of locating with the help of distance indices a <<refuge >> platform, invisible by immersion in a tank filled with opacified water. The device makes it possible to evaluate the reference memory of the animal (the platform remains in the same place on each day of the test). This test makes it possible to appreciate the mnestic performances dependent upon the functions of the hippocampus of the animals tested. In particular, this test makes it possible to discriminate between the performances of adult rats (10 months) and those of old rats (30 months). The hippocampus is a cerebral structure the functions of which are altered prematurely in Alzheimer's disease. The Morris pool test is therefore particularly recognised by experts in the field as making it possible to appreciate the pharmacological properties of compounds intended to treat Alzheimer's disease and other diseases associated with a cognitive deficit.

Old rats treated with etazolate (3 mg/kg and 10 mg/kg) administered orally and rats treated by the vehicle were used for this study. The performances of these animals in the Morris pool test were compared to those of a control group of adult rats.

Treatment with 3 mg/kg etazolate slightly improves the performances of old rats. Treatment with 10 mg/kg etazolate importantly brings the performances of old animals closer to those of adult animals.

This result indicates that etazolate improves the mnestic and cognitive properties dependent upon the hippocampus, making it possible to reduce the deficits of performance linked to age. This result qualifies etazolate for the treatment of cognitive problems linked to age such as Alzheimer's disease in particular.

### Example 6

### Clinical Use in Human Beings

This example describes the conditions for use in man of etazolate for the treatment of neurodegenerative diseases. This example illustrates the therapeutical potential of the invention and its conditions for use in man.

In this study, single, increasing doses of etazolate (0.5, 1, 2, 5, 10 and 20 mg) were administered orally in the form of capsules dosed at 0.5 and 5 mg to different and sequential groups of eight young, healthy, volunteer subjects of the male sex. This study was carried out in just one centre, double-blind, and two of the eight subjects were given a placebo. The parameters evaluated were clinical (appearance of adverse effects, of clinical signs, change in arterial pressure or heart rate), electrocardiographic (ECG recording) and biological tolerance (hematology and sanguineous biochemistry, urinary examination) for the 24 hrs following administration of the product. A plasmatic dosage of the product was carried out in each subject at different times before and after administration of the product (0.25-0.50-1.00-1.50-2.00-3.00-4.00-5.00-6.00-8.00-10.00-12.00 and 24.00 hours). A urinary dosage of the product was also carried out from urines collected before and after administration of the product (4, 4-8, 8-12 and 12-24 hours).

At the end of this phase of administering increasing doses, an additional group of six subjects receives on two occasions a dose of etazolate: on an empty stomach, and during a meal rich in fat. The objective of this second part is to compare the development of blood rates of the product between the two administration conditions. The parameters evaluated are clinical (appearance of adverse effects, clinical signs, change in arterial pressure or heart rate), electrocardiographic (ECG recording) and biological tolerance (hematological and sanguineous biochemistry, urinary examination) for the 24 hours following administration of the product. A plasmatic dosage of the product is carried out in each subject at different times before and after administration of the product (0.25-0.50-1.00-1.50-2.00-3.00-4.00-5.00-6.00-8.00-10.00-12.00 and 24.00 hours). A urinary dosage of the product is also carried out from urines collected before and after administration of the product (4, 4-8, 8-12 and 12-24 hours).

A gastro-resistant capsule is also developed for this product such as to be able to use it in clinical studies in humans.

The results obtained during the first study phase of increasing doses showed that etazolate was well tolerated and did not involve any secondary effects. In addition, the plasmatic dosages confirmed in humans the good absorption of the product at strong doses.

1-6. (canceled)

7. A method of treating cognitive deficits in a patient having a neurodegenerative disease, the method comprising administering to the patient an effective amount of a compound of the pyrazolopyridine family.

8. The method of claim 7, wherein the compound is a substituted or substituted compound of formula (I)

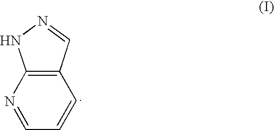

9. The method of claim 8, wherein the compound is etazolate or tracazolate.

10. The method of claim 7, wherein the compound is selected from the following compounds:
Ethylic ester of 4-butylamino-1-ethyl-6-methyl-1H-pyrazolo[3,4-b]pyridine-5-carboxylic acid (tracazolate),
Ethylic ester of 4-butylamino-1-ethyl-1H-pyrazolo[3,4-b]pyridine-5-carboxylic acid
1-(4-amino-pyrazolo[3,4-b]pyridin-1-yl)-□-D1-deoxy-ribofuranose
Ethylic ester of 1-ethyl-4-(N′-isopropylidene-hydrazino)-1H-pyrazolo[3,4-b]pyridine-5-carboxylic acid (SQ 20009),
4-amino-6-methyl-1-n-pentyl-1H-pyrazolo[3,4-b]pyridine
Ethylic ester of 4-Amino-1-ethyl-6-methyl-1H-pyrazolo[3,4-b]pyridine-5-carboxylic acid (desbutyl tracacolate),
4-amino-1-pentyl-1H-pyrazolo[3,4-b]pyridine-5-carboxamide,
Ethylic ester of 1-ethyl-6-methyl-4-methylamino-1H-pyrazolo[3,4-b]pyridine-5-carboxylic acid,
Ethylic ester of 4-amino-6-methyl-1-propyl-1H-pyrazolo[3,4-b]pyridine-5-carboxylic acid,
Ethylic ester of 1-ethyl-4-ethylamino-6-methyl-1H-pyrazolo[3,4-b]pyridine-5-carboxylic acid,
Ethylic ester of 4-amino-1-butyl-6-methyl-1H-pyrazolo[3,4-b]pyridine-5-carboxylic acid,
5-(4-amino-pyrazolo[3,4-b]pyridin-1-yl)-2-hydroxymethyl-tetrahydro-furan-3-ol, allylic ester of 1-allyl-4-amino-6-methyl-1H-pyrazolo[3,4-b]pyridine-5-carboxylic acid,
4-amino-6-methyl-1-pentyl-1H-pyrazolo[3,4-b]pyridine-5-carboxylic acid,
ethylic ester of 4-amino-1-ethyl-3,6-dimethyl-1H-pyrazolo[3,4-b]pyridine-5-carboxylic acid,
ethylic ester of 4-dimethylamino-1-ethyl-6-methyl-1H-pyrazolo[3,4-b]pyridine-5-carboxylic acid,
ethylic ester of 1-ethyl-6-methyl-4-propylamino-1H-pyrazolo[3,4-b]pyridine-5-carboxylic acid,
ethylic ester of 4-amino-1-pentyl-1H-pyrazolo[3,4-b]pyridine-5-carboxylic acid,
ethylic ester of 4-amino-6-methyl-1-pent-4-ynyl-1H-pyrazolo[3,4-b]pyridine-5-carboxylic acid,
4-amino-1-but-3-enyl-1H-pyrazolo[3,4-b]pyridine-5-allylamide,
4-amino-1-pentyl-1H-pyrazolo[3,4-b]pyridine-5-isopropylamide,
4-amino-1-pentyl-N-n-propyl-1H-pyrazolo-[3,4-b]pyridine-5-carboxamide,
allylic ester of 4-amino-1-butyl-6-methyl-1H-pyrazolo[3,4-b]pyridine-5-carboxylic acid,
ethylic ester of 4-amino-6-methyl-1-pent-3-ynyl-1H-pyrazolo[3,4-b]pyridine-5-carboxylic acid,
4-amino-1-pentyl-1H-pyrazolo[3,4-b]pyridine-5-prop-2-ynylamide
allylic ester of 4-amino-1-(3-methyl-butyl)-1H-pyrazolo[3,4-b]pyridine-5-carboxylic acid,
4-amino-1-pentyl-1H-pyrazolo[3,4-b]pyridine-5-N-(2-propenyl)carboxamide,
allylic ester of 4-amino-1-pentyl-1H-pyrazolo[3,4-b]pyridine-5-carboxylic acid,
4-amino-1-pentyl-1H-pyrazolo[3,4-b]pyridine-5-butylamide,
allylic ester of 4-amino-1-but-3-ynyl-6-methyl-1H-pyrazolo[3,4-b]pyridine-5-carboxylic acid,
allylic ester of 4-amino-1-but-3-enyl-6-methyl-1H-pyrazolo[3,4-b]pyridine-5-carboxylic acid,
4-amino-6-methyl-1-pentyl-1H-pyrazolo[3,4-b]pyridine-5-allylamide,
allylic ester of 4-amino-6-methyl-1-pentyl-1H-pyrazolo[3,4-b]pyridine-5-carboxylic acid,
allylic ester of 4-amino-6-methyl-1-(3-methyl-butyl)-1H-pyrazolo[3,4-b]pyridine-5-carboxylic acid,
isobutylic ester of 4-amino-6-methyl-1-pentyl-1H-pyrazolo[3,4-b]pyridine-5-carboxylic acid,
4-amino-6-methyl-1-pentyl-1H-pyrazolo[3,4-b]pyridine-5-butylamide,
allylic ester of 4-amino-6-methyl-1-(3-methyl-but-2-enyl)-1H-pyrazolo[3,4-b]pyridine-5-carboxylic acid,
4-amino-1-pentyl-1H-pyrazolo[3,4-b]pyridine-5-cyclopropylamide,
ethyl 4-amino-1-pentyl-1H-pyrazolo[3,4-b]pyridine-5-hydroxamate,
prop-2-ynylic ester of 4-amino-6-methyl-1-pentyl-1H-pyrazolo[3,4-b]pyridine-5-carboxylic acid,
allylic ester of 4-amino-6-methyl-1-pent-4-ynyl-1H-pyrazolo[3,4-b]pyridine-5-carboxylic acid,
allylic ester of 4-amino-6-methyl-1-pent-4-enyl-1H-pyrazolo[3,4-b]pyridine-5-carboxylic acid,
4-amino-1-pent-3-ynyl-1H-pyrazolo[3,4-b]pyridine-5-propylamide,
4-amino-1-pentyl-1H-pyrazolo[3,4-b]pyridine-5-cyclopropylmethyl-amide,
2-methyl-allylic ester of 4-amino-6-methyl-1-pentyl-1H-pyrazolo[3,4-b]pyridine-5-carboxylic acid,
4-Amino-1-pent-3-ynyl-1H-pyrazolo[3,4-b]pyridine-5-allylamide (ICI 190,622),
4-amino-1-pent-4-ynyl-N-2-propenyl-1H-pyrazolo[3,4-b]pyridine-5-carboxamide,
4-amino-1-pent-3-ynyl-1H-pyrazolo[3,4-b]pyridine-5-prop-2-ynylamide,
4-amino-1-pentyl-1H-pyrazolo[3,4-b]pyridine-5-but-2-ynylamide,
allylic ester of 4-amino-6-methyl-1-pent-3-ynyl-1H-pyrazolo[3,4-b]pyridine-5-carboxylic acid,
allylic ester of 4-amino-1-(2-cyclopropyl-ethyl)-6-methyl-1H-pyrazolo[3,4-b]pyridine-5-carboxylic acid,
allylic ester of 4-amino-1-hex-5-ynyl-6-methyl-1H-pyrazolo[3,4-b]pyridine-5-carboxylic acid,
4-amino-1-pent-3-ynyl-1H-pyrazolo[3,4-b]pyridine-5-cyclopropylmethyl-amide,
but-3-enylic ester of 4-amino-6-methyl-1-pentyl-1H-pyrazolo[3,4-b]pyridine-5-carboxylic acid,
cyclopropylmethylic ester of 4-amino-6-methyl-1-pentyl-1H-pyrazolo[3,4-b]pyridine-5-carboxylic acid,
4-butylamino-1-pentyl-1H-pyrazolo[3,4-b]pyridine-5-allylamide,
2-cyclopropyl-ethylic ester of 4-amino-6-methyl-1-pentyl-1H-pyrazolo[3,4-b]pyridine-5-carboxylic acid,
cyclopropylmethylic ester of 4-amino-6-methyl-1-pent-3-ynyl-1H-pyrazolo[3,4-b]pyridine-5-carboxylic acid,
cyclopropylmethylic ester of 4-amino-6-methyl-1-pent-4-ynyl-1H-pyrazolo[3,4-b]pyridine-5-carboxylic acid,
ethylic ester of 4-amino-1-benzyl-6-methyl-1H-pyrazolo[3,4-b]pyridine-5-carboxylic acid,
4-amino-1-pentyl-1H-pyrazolo[3,4-b]pyridine-5-benzylamide,
4-amino-1-pentyl-1H-pyrazolo[3,4-b]pyridine-5-phenylamide,
benzylic ester of 4-amino-6-methyl-1-pentyl-1H-pyrazolo[3,4-b]pyridine-5-carboxylic acid,
4-Azido-1-□-D-ribofuranosylpyrazolo[3,4-b]pyridine,
1-pent-3-ynyl-N-2-propenyl-4-propionamido-1H-pyrazolo[3,4-b]pyridine-5-carboxamide,
2-(4-amino-pyrazolo[3,4-b]pyridin-1-yl)-5-hydroxymethyl-tetrahydro-furan-3,4-diol,
2-(6-methyl-1H-pyrazolo[3,4-b]pyridin-4-ylamino)-ethanol,
3-(6-methyl-1H-pyrazolo[3,4-b]pyridin-4-ylamino)-propan-1-ol,
propylic ester of 3-(6-methyl-1H-pyrazolo[3,4-b]pyridin-4-ylamino)-acetic acid,
ethylic ester of 2-(6-methyl-1H-pyrazolo[3,4-b]pyridin-4-ylamino)-propionic acid,
ethylic ester of 2-(6-methyl-1H-pyrazolo[3,4-b]pyridin-4-ylamino)-pentanoic acid,
ethylic ester of 2-(6-methyl-1H-pyrazolo[3,4-b]pyridin-4-ylamino)-benzoic acid,
propylic ester of 3-(6-methyl-1H-pyrazolo[3,4-b]pyridin-4-ylamino)-pentanoic acid,
N-benzylidene-N′-(3-methyl-1-phenyl-1H-pyrazolo[3,4-b]pyridin-4-yl)-hydrazine,
N-furan-2-ylmethylene-N′-(3-methyl-1-phenyl-1H-pyrazolo[3,4-b]pyridin-4-yl)-hydrazine,
N-(4-fluoro-benzylidene)-N′-(3-methyl-1-phenyl-1H-pyrazolo[3,4-b]pyridin-4-yl)-hydrazine,
N-(3-furan-2-yl-allylidene)-N′-(3-methyl-1-phenyl-1H-pyrazolo[3,4-b]pyridin-4-yl)-hydrazine,
N-(4-methoxy-benzylidene)-N′-(3-methyl-1-phenyl-1H-pyrazolo[3,4-b]pyridin-4-yl)-hydrazine,
4-[(3-methyl-1-phenyl-1H-pyrazolo[3,4-b]pyridin-4-yl)-hydrazonomethyl]-benzonitrile,
N-benzo[1,3]dioxol-5-ylmethylene-N′-(3-methyl-1-phenyl-1H-pyrazolo[3,4-b]pyridin-4-yl)-hydrazine,
N-(3-methyl-1-phenyl-1H-pyrazolo[3,4-b]pyridin-4-yl)-N′-(4-nitro-benzylidene)-hydrazine,
N-(3-methyl-1-phenyl-1H-pyrazolo[3,4-b]pyridin-4-yl)-N′-(2-nitro-benzylidene)-hydrazine,
N-(3-methyl-1-phenyl-1H-pyrazolo[3,4-b]pyridin-4-yl)-N′-(4-trifluoromethyl-benzylidene)-hydrazine,
N-(3-methyl-1-phenyl-1H-pyrazolo[3,4-b]pyridin-4-yl)-N′-(5-nitro-furan-2-ylmethylene)-hydrazine,
N-(3-methyl-1-phenyl-1H-pyrazolo[3,4-b]pyridin-4-yl)-N′-(2-trifluoromethyl-benzylidene)-hydrazine,
N-(3-methyl-1-phenyl-1H-pyrazolo[3,4-b]pyridin-4-yl)-N′-(6-nitro-benzo[1,3]dioxol-5-ylmethylene)-hydrazine,
4-(3-chloro-4-methoxy-benzylamino)-1-ethyl-1H-pyrazolo[3,4-b]pyridine-5-carboxylic acid,
4-(3-chloro-4-methoxy-benzylamino)-1-ethyl-1H-pyrazolo[3,4-b]pyridine-5-(pyridin-4-ylmethyl)-amide,
4-(3-chloro-4-methoxy-benzylamino)-1-ethyl-1H-pyrazolo[3,4-b]pyridine-5-(tetrahydro-furan-2-ylmethyl)-amide,
4-(3-chloro-4-methoxy-benzylamino)-1-ethyl-1H-pyrazolo[3,4-b]pyridine-5-(5-hydroxy-pentyl)-amide,
4-(3-chloro-4-methoxy-benzylamino)-1-ethyl-1H-pyrazolo[3,4-b]pyridine-5-[3-(2-oxo-pyrrolidine-1-yl)-propyl]-amide,
ethylic ester of 4-tert-butylamino-1-(2-chloro-2-phenyl-ethyl)-1H-pyrazolo[3,4-b]pyridine-5-carboxylic acid,
ethylic ester of 1-(2-chloro-2-phenyl-ethyl)-4-cyclopropylamino-1H-pyrazolo[3,4-b]pyridine-5-carboxylic acid,
ethylic ester of 1-(2-chloro-2-phenyl-ethyl)-4-propylamino-1H-pyrazolo[3,4-b]pyridine-5-carboxylic acid,
ethylic ester of 1-(2-chloro-2-phenyl-ethyl)-4-phenylamino-1H-pyrazolo[3,4-b]pyridine-5-carboxylic acid,
ethylic ester of 4-butylamino-1-(2-chloro-2-phenyl-ethyl)-1H-pyrazolo[3,4-b]pyridine-5-carboxylic acid,
ethylic ester of 1-(2-chloro-2-phenyl-ethyl)-4-(2-ethoxy-ethylamino)-1H-pyrazolo[3,4-b]pyridine-5-carboxylic acid,
ethylic ester of 4-benzylamino-1-(2-chloro-2-phenyl-ethyl)-1H-pyrazolo[3,4-b]pyridine-5-carboxylic acid, and
ethylic ester of 1-(2-chloro-2-phenyl-ethyl)-4-phenethylamino-1H-pyrazolo[3,4-b]pyridine-5-carboxylic acid.

11. The method of claim 7, for treating cognitive deficit in a patient having a neurodegenerative disease selected from Alzheimer's disease, vascular dementia, Parkinson's disease and Huntington's chorea.

12. The method of claim 7, wherein the composition is administered orally or systemically.

13. A method of improving perceptive cognition in a patient having a neurodegenerative disease, the method comprising administering to the patient an effective amount of etazolate.

14. The method of claim 13, wherein the patient has an Alzheimer's disease or vascular dementia.
